(19) United States
(12) Patent Application Publication
(10) Pub. No.: US 2009/0016983 A1
(43) Pub. Date: Jan. 15, 2009

(54) Hair conditioning composition comprising polysaccharide polymer and aminosilicone

(76) Inventors: Natsumi Komure, Ashiya (JP); Golam Faruque Khan, Darien, CT (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/825,795

(22) Filed: Jul. 9, 2007

(51) Int. Cl. A61K 8/73; A61P 43/00; A61Q 5/02

(52) U.S. Cl. 424/70.122; 424/70.13

(57) ABSTRACT

Disclosed is a hair conditioning composition comprising: a cationic or nonionic polysaccharide polymer; an aminosilicone; a cationic surfactant; and a high melting point fatty compound. The composition of the present invention can provide hair volume-up benefit and ease-to-rinse feel, while providing improved conditioning benefits. The present invention also relates to a use of the above composition for hair volume-up.

### FIELD OF THE INVENTION

The present invention relates to a hair conditioning composition comprising: a cationic or nonionic polysaccharide polymer; an aminosilicone; a cationic surfactant; and a high melting point fatty compound. The composition of the present invention can provide hair volume-up benefit and ease-to-rinse feel, while providing improved conditioning benefits.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits.

Hair conditioning compositions comprising the conditioning agent described above can provide conditioning benefits such as softness and moisturized feel on dry hair, however, also weigh down the hair. The weighed down hair gives an appearance of reduced bulk hair volume. For consumers who desire hair volume-up such as consumers having fine hair, the effect of hair weighing down is not desirable. The term “hair volume-up” as used herein is not equal to fly-away hair. Fly-away hair is due to the increased level of static, and represents volume increase of only very minor amount of the hair as a whole, and is not desirable. On the other hand, hair volume-up as used herein relates to increase of the bulk of the hair volume. Consumers having fine hair have the desire to achieve hair volume-up while controlling undesirable fly-away of the hair. Thus, there is a need for hair conditioning compositions providing hair volume-up benefit while providing conditioning benefits.

There also exists a need for hair conditioning compositions which provide improved ease-to-rinse feel, while maintaining conditioning benefits especially when forming a gel matrix comprised of cationic surfactants and high melting point fatty compounds. Hair conditioning compositions containing a gel matrix provide slippery feel during rinsing the hair, even after rinsing the hair. Ease-to-rinse feel are, for example, fast reduced slippery feel, and/or increased clean feel after starting to rinse the hair. Such reduced slippery feel, and/or increased clean feel can be a signal for consumers to stop rinsing the hair conditioning compositions. Consumers who prefer such signals may keep rinsing the hair treated with hair conditioning composition by a large amount of water until they feel the signals. However, such rinsing activity provides less deposition of conditioning agents on the hair, thus less conditioning benefits to the dry hair. Thus, there remains a need for hair conditioning compositions which provides such signals faster after starting to rinse the hair, while remaining sufficient amount of deposition of conditioning agents on the hair and/or maintaining conditioning benefits.

Japanese Patent Laid-open 2004-143093 discloses a hair conditioning composition comprising: condensed polyalcohols having a viscosity of 4-100 Pa·s at 25° C.; oily agents having a melting point of 30-55° C.; silicones having amino groups or derivatives thereof; nonionic surfactants having polyoxyalkylene adjunctions; and saturated fatty alcohols, the composition being said to provide sufficient moisturization and emollient to damaged hair, while providing excellent feel such as less stickiness, less unnatural heaviness, better finger combing feel, and softness. Japanese Patent Laid-open 2004-143093 also disclose, in Example 21, a hair conditioning composition containing: hydroxypropyl guar; cetostearyl alcohol; stearyl trimethyl ammonium chloride; amino-modified silicone with tradename SM8704C supplied from Dow Corning Toray.

However, it has been found that it is still not easy to obtain hair conditioning compositions which provide hair volume-up benefit and ease-to-rinse feel, while providing improved conditioning benefits.

Based on the foregoing, there remains a need for hair conditioning compositions providing hair volume-up benefit and ease-to-rinse feel, while providing improved conditioning benefits.

None of the existing art provides all of the advantages and benefits of the present invention.

### SUMMARY OF THE INVENTION

The present invention is directed to a hair conditioning composition comprising by weight:
(a) from about 0.05% to about 10% of a cationic or nonionic polysaccharide polymer having a molecular weight of about 1,000,000 or more, wherein the polysaccharide polymer is selected from a cellulose, a guar gum, a cassia gum, a starch, and derivatives thereof;
(b) from about 0.1% to about 10% of an aminosilicone having a formula:

(R₁)ₐG₃₋ₐ-Si-(-OSiG₂)ₙ-O—SiG₃₋ₐ(R₁)ₐ

wherein G is hydrogen, phenyl, hydroxy, or C₁-C₈ alkyl; a is an integer having a value from 1 to 3; n is a number from 1 to 2,000; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion;
(c) from about 0.1% to about 10% by weight of the composition of a cationic surfactant;
(d) from about 0.1% to about 20% by weight of the composition of a high melting point fatty compound; and
(e) an aqueous carrier.

The conditioning compositions of the present invention provide hair volume-up benefit and ease-to-rinse feel, while providing improved conditioning benefits.

The present invention is also directed to use of a hair conditioning composition for hair volume-up, wherein the hair conditioning composition comprising by weight:
(a) from about 0.05% to about 10% of a cationic or nonionic polysaccharide polymer having a molecular weight of about 1,000,000 or more, wherein the polysaccharide polymer is selected from a cellulose, a guar gum, a cassia gum, a starch, and derivatives thereof;
(b) from about 0.1% to about 10% by weight of the composition of a cationic surfactant;
(c) from about 0.1% to about 20% by weight of the composition of a high melting point fatty compound; and
(d) an aqueous carrier;
   and wherein the hair conditioning composition preferably further comprises: from about 0.1% to about 10% of an aminosilicone having a formula:

(R₁)ₐG₃₋ₐ-Si-(-OSiG₂)ₙ-O—SiG₃₋ₐ(R₁)ₐ

wherein G is hydrogen, phenyl, hydroxy, or C₁-C₈ alkyl; a is an integer having a value from 1 to 3; n is a number from 1 to 2,000; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Composition

It is believed that; by the use of the polysaccharide polymers having a molecular weight of about 1,000,000 or more and the aminosilicone having the specific formula, together with other required elements, the conditioning compositions of the present invention provide hair volume-up benefit and ease-to-rinse feel, while providing improved conditioning benefits.

Preferably, when containing cationic surfactants and/or gel matrix formed by cationic surfactants and high melting point fatty compounds, the composition of the present invention is substantially free of anionic surfactants and anionic polymers, in view of avoiding undesirable interaction with cationic surfactants and/or in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

### Polysaccharide Polymer

The composition of the present invention comprises a polysaccharide polymer. The polymers are included in the composition at a level by weight of from about 0.05% to about 10%, preferably from about 0.08% to about 5%, more preferably from about 0.1% to about 1%, in view of rheology.

The polymers useful herein are cationic or nonionic, in view of avoiding undesirable interaction with cationic surfactants. The polymers useful herein are preferably nonionic, in view of ease-to-rinse feel.

The polymers useful herein are those having a molecular weight of about 1,000,000 or more, preferably 1,500,000 or more, more preferably 2,000,000 or more, in view of rheology and hair volume-up benefit. The molecular weight is generally up to about 5,000,000, preferably to about 4,000,000 in view of water solubility.

The polymers useful herein are water-soluble. In the present invention, “water-soluble polymers” means that the polymers have a solubility of greater than 0.1 g/100 g water, preferably 0.3 g/100 g water at 25° C., more preferably 0.5 g/100 g water at 25° C.

The polymers useful herein are those selected from a cellulose, a guar gum, a cassia gum, a starch, and derivatives thereof. Such polymers, include, for example, cellulose and its derivatives such as cellulose ethers including hydroxyethylcellulose and hydroxypropylcellulose, hydrophobically modified cellulose ethers such as cetyl hydroxyethylcellulose which is supplied, for example, by Hercules with a tradename Polysurf 67, quaternized celluloses, and hydrophobically modified cationic celluloses; guar polymers including cationic guar polymers and nonionic guar polymers such as Guar Gum 2-hydroxypropyl ether which is supplied, for example, by Rhodia with a tradename Jaguar HP-105; and cassia gums and cationic cassia gums.

### Nonionic Guar Gum Derivatives

In the composition, nonionic guar gum derivatives are preferably used among a variety of the above polymers, in view of hair volume-up benefit. Such derivatives useful herein are guar gums modified with C1-C6 hydroxyalkyl or alkyl groups and with fatty chains having from 8 to 30 carbon atoms such as linear or branched alkyl or alkenyl groups. It is believed that, bulkier backbones of guar gum compared to those of other polymers such as cellulose can provide a thicker polymer layer on the hair and provide hair volume-up benefit. It is also believed that, by having such substitutions attached to a guar gum backbone, the guar gum derivatives can provide a further thicker polymer layer on the hair and provide hair volume-up benefit. Among the above substitutions, preferred are C1-C6 hydroxyalkyl groups, more preferred are those selected from the group consisting of hydroxymethyl, hydroxyethyl, hydroxypropyl and hydroxybutyl groups, and still more preferred are hydroxypropyl groups. It is believed that nonionic substitution can deliver better ease-to-rinse feel, compared to cationic substitutions. The degree of hydroxyalkylation, which corresponds to the number of alkylene oxide molecules consumed by the number of free hydroxyl functions present on the guar gum, preferably ranges from 0.2 to 1.2, and more preferably ranges from 0.4 to 1.2. Commercially available examples of highly preferred nonionic guar derivatives include, for example, hydroxypropyl guar gum having a molecular weight of about 2,500,000, having a degree of hydroxyalkylation of about 0.6 having a tradename Jaguar HP-105 available from Rhodia.

### Aminosilicone

The compositions of the present invention comprise an amino silicone. The aminosilicone useful herein are those which conform to the general formula (I):

(R₁)ₐG₃₋ₐ-Si-(-OSiG₂)ₙ-O—SiG₃₋ₐ(R₁)ₐ

wherein G is hydrogen, phenyl, hydroxy, or C₁-C₈ alkyl, preferably methyl; a is an integer having a value from 1 to 3, preferably 1; n is a number from 1 to about 2,000, preferably from about to about 100 to 2,000, more preferably from 300 to 1,800; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion. L is preferably —N(CH₃)₂ or —NH₂.

It is believed that; the amino silicone of the above formula can provide a good balance among hair volume-up, easy-to-rinse feel and conditioning benefits compared to other silicones, when used in the composition of the present invention. Such other silicones are, for example, silicones without amino substitutions and graft aminosilicones. The amino silicone of the above formula is used at levels by weight of the composition of from about 0.1% to about 10%, preferably from about 0.1% to about 7%, more preferably from about 0.2% to about 5%.

One highly preferred amino silicones are those corresponding to formula (I) wherein a=1, q=3, G=methyl, n is preferably from about 1400 to about 1700, more preferably about 1600; and L is —N(CH₃)₂ or —NH₂, more preferably —NH₂. Another highly preferred amino silicones are those corresponding to formula (I) wherein a=1, q=3, G=methyl, n is preferably from about 400 to about 800, more preferably from about 500 to about 600; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

### Cationic Surfactant System

The composition of the present invention comprises a cationic surfactant system. The cationic surfactant system can be one cationic surfactant or a mixture of two or more cationic surfactants. The cationic surfactant system is included in the composition at a level by weight of from about 0.1% to about 10%, preferably from about 0.5% to about 8%, more preferably from about 1% to about 5%, still more preferably from about 1.4% to about 4%, in view of balance among ease-to-rinse feel, rheology and wet conditioning benefits.

A variety of cationic surfactants including mono- and di-alkyl chain cationic surfactants can be used in the compositions of the present invention. Among them, preferred are mono-alkyl chain cationic surfactants in view of providing desired gel matrix and wet conditioning benefits. The mono-alkyl cationic surfactants are those having one long alkyl chain which has from 12 to 22 carbon atoms, preferably from 16 to 22 carbon atoms, more preferably C18-22 alkyl group, in view of providing balanced wet conditioning benefits. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Such mono-alkyl cationic surfactants include, for example, mono-alkyl quaternary ammonium salts and mono-alkyl amines. Mono-alkyl quaternary ammonium salts include, for example, those having a non-functionalized long alkyl chain. Mono-alkyl amines include, for example, mono-alkyl amidoamines and salts thereof.

Mono-long alkyl quaternized ammonium salts useful herein are those having the formula (II):

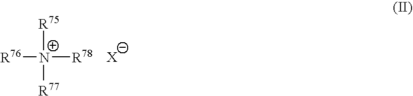

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Examples of preferred mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt. Among them, highly preferred are behenyl trimethyl ammonium salt and stearyl trimethyl ammonium salt.

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Although the mono-alkyl chain cationic surfactants are preferred, other cationic surfactants such as di-alkyl chain cationic surfactants may also be used alone, or in combination with the mono-alkyl chain cationic surfactants. Such di-alkyl chain cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound is included in the composition at a level of from about 0.1% to about 20%, preferably from about 1% to about 15%, more preferably from about 1.5% to about 8% by weight of the composition, in view of providing improved conditioning benefits such as slippery feel during the application to wet hair, softness and moisturized feel on dry hair.

### Gel Matrix

The composition of the present invention comprises a gel matrix. The gel matrix comprises a cationic surfactant, a high melting point fatty compound, and an aqueous carrier.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

### Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate; and antidandruff agents such as zinc pyrithione and salicylic acid.

### Additional Silicone Compounds

The compositions of the present invention may contain additional silicone compound, in addition to those described above under the title “AMINOSILICONE”. The additional silicone compounds herein can be contained at a level by weight of the composition of preferably from about 0.1% to about 10%, more preferably from about 0.1% to about 5%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Coming Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

The above polyalkylsiloxanes are available, for example, as a mixture with silicone compounds having a lower viscosity. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

### Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Particularly useful citrate ester oils herein include triisocetyl citrate, triisostearyl citrate, and trioctyldodecyl citrate. Particularly useful glyceryl ester oils herein include triisostearin, triolein, and trilinolein. Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

### Cationic Conditioning Polymer

Cationic conditioning polymers useful herein are those having an average molecular weight of at least about 5,000, typically from about 10,000, preferably from about 100,000 to about 2 million. Cationic conditioning polymers useful herein are not polymers described above under the tile “POLYSACCHARIDE POLYMER”.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol.

### Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:
(i) after shampooing hair, applying to the hair an effective amount of the conditioning compositions for conditioning the hair; and
(ii) then rinsing the hair.

It is preferred to use the composition of the present invention for hair volume-up. When used in a product for hair volume-up, it is preferred that such product has at least one depiction corresponding to hair volume-up benefit. Such depiction includes, for example, “Volume-up”, “Shear Volume”, “Volumizing”, “Hair Fullness”, “Hair Body”, “Thicker Hair”, “Not-Weighing Down Hair”, “Expanding”, “Lighter Hair”, “Bouncy”, “Airy”, and “Lift From Roots”.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Polysaccharide polymer-1 *1 | 0.35 | 0.35 | 0.35 | — | 0.35 | 0.3 | — | — |
| Polysaccharide polymer-2 *2 | — | — | — | 0.1 | — | — | — | — |
| Polysaccharide polymer-3 *3 | — | — | — | — | — | — | 0.05 | — |
| Polysaccharide polymer-4 *4 | — | — | — | — | — | — | — | 0.4 |
| Aminosilicone-1 *5 | 0.5 | 1.0 | 0.75 | 2.5 | 1.5 | 0.5 | 1.0 | 0.5 |
| Stearamidopropyldimethylamine | 1.0 | 1.0 | — | — | — | — | 2.0 | 2.1 |
| Ditallow dimethyl ammonium | 0.75 | — | — | — | — | — | — | — |
| chloride | | | | | | | | |
| Distearyl dimethyl ammonium | — | 0.75 | — | — | — | — | — | — |
| chloride | | | | | | | | |
| Dicetyl dimethyl ammonium chloride | — | — | 0.35 | — | — | — | — | — |
| Behenyl trimethyl ammonium | — | — | — | 2.3 | — | — | — | — |
| chloride | | | | | | | | |
| Behenyl trimethyl ammonium | — | — | 1.1 | — | 1.8 | — | — | — |
| methosulfate | | | | | | | | |
| Behenamidopropyldimethylamine | — | — | — | — | — | 2.3 | — | — |
| Cetyl alcohol | 1.3 | 1.5 | 0.9 | 1.9 | 1.5 | 2.5 | 2.5 | 2.7 |
| Stearyl alcohol | 1.1 | 1.2 | 2.3 | 4.6 | 3.7 | 4.5 | 4.5 | 4.8 |
| Oleyl alcohol | 0.25 | 0.2 | — | — | — | — | — | — |
| Polysorbate-60 | 0.1 | 0.1 | — | — | — | — | — | — |
| Glycerylmonostearate | 0.25 | 0.25 | — | — | — | — | — | — |
| L-Glutamic acid | — | — | — | — | — | 0.64 | 0.64 | 0.69 |
| Citric acid | 0.13 | 0.13 | — | — | — | — | — | 0.1 |
| EDTA | 0.1 | 0.1 | — | — | — | 0.1 | 0.1 | 0.1 |
| Disodium EDTA | — | — | 0.13 | 0.13 | 0.13 | — | — | — |
| Preservatives | 0.4 | 0.4 | 0.4 | 0.4 | 0.4 | 0.4 | 0.4 | 0.4 |
| Perfume | 0.35 | 0.25 | 0.35 | 0.4 | 0.25 | 0.25 | 0.25 | 0.25 |
| Panthenol | 0.05 | — | 0.05 | — | — | — | 0.05 | 0.05 |
| Panthenyl ethyl ether | 0.05 | — | 0.05 | — | — | — | 0.05 | 0.05 |

| | |
|---|---|
| Deionized Water | q.s. to 100% |

Definitions of Components
*1 Polysaccharide polymer-1: hydroxypropyl guar polymer having a molecular weight of about 2,500,000 and having a degree of substitution of 0.6, available from Rhodia with a tradename Jaguar HP-105.
*2 Polysaccharide polymer-2: guar hydroxypropyltrimonium chloride having a molecular weight of 2,200,000 and having a charge density of 0.6, available from Rhodia with a tradename Jaguar C17.
*3 Polysaccharide polymer-3: guar hydroxypropyltrimonium chloride having a molecular weight of 1,200,000 and having a charge density of 0.7, available from Rhodia with a tradename Jaguar Excel.
*4 Polysaccharide polymer-4: hydroxypropyl guar having a molecular weight of 1,400,000 and having a degree of substitution of 0.4 available from Aqualon/Hercules with a tradename N-Hance HP-40.
*5 Aminosilicone-1: Terminal aminosilicone which is available from GE having a viscosity of about 10,000 mPa · s, and having following formula: (R₁)ₐG₃₋ₐ—Si—(—OSiG₂)ₙ—O—SiG₃₋ₐ(R₁)ₐ wherein G is methyl; a is an integer of 1; n is a number from 400 to about 600; R₁ is a monovalent radical conforming to the general formula C_{q}H_{2q}L, wherein q is an integer of 3 and L is —NH₂.

### Method of Preparation

The conditioning compositions of “Ex. 1” through “Ex. 8” as shown above can be prepared by any conventional method well known in the art. They are suitably made as follows:

Polymeric materials are added to water with agitation, and heated to about 80° C. Cationic surfactants and high melting point fatty compounds are added to the mixture at about 80° C. with agitation. The mixture is cooled down to about 55° C. Aminosilicone and, if included, additional components such as perfumes and preservatives are added to the mixture with agitation. Then the mixture is cooled down to room temperature.

Examples 1 through 8 are hair conditioning compositions of the present invention which are particularly useful for rinse-off use. The embodiments disclosed and represented by the previous “Ex. 1” through “Ex. 8” have many advantages. For example, they provide hair volume-up benefit and ease-to-rinse feel, while providing improved conditioning benefits.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the same term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising by weight:
(a) from about 0.05% to about 10% of a cationic or nonionic polysaccharide polymer having a molecular weight of about 1,000,000 or more, wherein the polysaccharide polymer is selected from a cellulose, a guar gum, a cassia gum, a starch, and derivatives thereof;
(b) from about 0.1% to about 10% of an aminosilicone having a formula:
(R₁)ₐG₃₋ₐ-Si-(-OSiG₂)ₙ-O—SiG₃₋ₐ(R₁)ₐ
wherein G is hydrogen, phenyl, hydroxy, or C₁-C₈ alkyl; a is an integer having a value from 1 to 3; n is a number from 1 to 2,000; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion;
(c) from about 0.1% to about 10% by weight of the composition of a cationic surfactant;
(d) from about 0.1% to about 20% by weight of the composition of a high melting point fatty compound; and
(e) an aqueous carrier.

2. The hair conditioning composition of claim 1 wherein the aminosilicone having a formula:
(R₁)ₐG₃₋ₐ-Si-(-OSiG₂)ₙ-O—SiG₃₋ₐ(R₁)ₐ
wherein G is hydrogen, phenyl, hydroxy, or C₁-C₈ alkyl; a is 1; n is a number from 100 to 2,000; R₁ is a monovalent radical conforming to the general formula C_{q}H_{2q}L, wherein q is an integer having a value from 2 to 8 and L is —N(R₂)₂; wherein R₂ is hydrogen, phenyl, benzyl, or a saturated hydrocarbon radical; and A⁻ is a halide ion.

3. The hair conditioning composition of claim 2 wherein L is —N(CH₃)₂ or —NH₂.

4. The hair conditioning composition of claim 1 wherein the polymer has a molecular weight of about 1,500,000 or more.

5. The hair conditioning composition of claim 1 wherein the polymer is nonionic.

6. The hair conditioning composition of claim 1 wherein the polymer is selected from guar gum and derivatives thereof.

7. The hair conditioning composition of claim 1 wherein the polymer is selected from nonionic guar gum derivatives, wherein the nonionic guar gum derivatives are guar gums modified by C1-C6 hydroxyalkyl groups and have a molecular weight of about 1,500,000 or more.

8. The hair conditioning composition of claim 1 wherein the composition is substantially free of anionic surfactants and anionic polymers.

9. A use of a hair conditioning composition for hair volume-up, wherein the hair conditioning composition comprising by weight:
(a) from about 0.05% to about 10% of a cationic or nonionic polysaccharide polymer having a molecular weight of about 1,000,000 or more, wherein the polysaccharide polymer is selected from a cellulose, a guar gum, a cassia gum, a starch, and derivatives thereof;
(b) from about 0.1% to about 10% by weight of the composition of a cationic surfactant;
(c) from about 0.1% to about 20% by weight of the composition of a high melting point fatty compound; and
(d) an aqueous carrier.

10. A use of a hair conditioning composition of claim 9 wherein the hair conditioning composition further comprises: from about 0.1% to about 10% of an aminosilicone having a formula:
(R₁)ₐG₃₋ₐ-Si-(-OSiG₂)ₙ-O—SiG₃₋ₐ(R₁)ₐ
wherein G is hydrogen, phenyl, hydroxy, or C₁-C₈ alkyl; a is an integer having a value from 1 to 3; n is a number from 1 to 2,000; R₁ is a monovalent radical conforming to the general formula C_{q}H_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion.
